(19) United States
(12) Patent Application Publication
(10) Pub. No.: US 2023/0022013 A1
(43) Pub. Date: Jan. 26, 2023

(54) System, Method, and Apparatus for Monitoring Fermentation

(71) Applicant: Gulf Photonics, Inc., Oldsmar, FL (US)

(72) Inventors: Patrick Bennetts, St. Petersburg, FL (US); Craig Scott Vogeley, Palm Harbor, FL (US); Bradley Paul Fernandez, Oldsmar, FL (US); Christopher Thomas Bailey, Spring Hill, FL (US)

(73) Assignee: Gulf Photonics, Inc., Oldsmar, FL (US)

(21) Appl. No.: 17/382,457

(22) Filed: Jul. 22, 2021

(51) Int. Cl. G01N 21/31; G01N 33/14

(52) U.S. Cl. CPC G01N 21/314; G01N 33/143; G01N 33/146; G01N 2021/3148

(57) ABSTRACT

A system for reading and reporting data from a production of an alcoholic beverage includes a data capture device having a sensor end for placement within a liquid. The sensor end has at least two sensors for measuring data related to the liquid. A transmitter located within the data capture device is coupled to the at least two sensors and the transmitter periodically transmits the data related to the liquid. Color transmission or reflection of the liquid is used to measure clarity, color of the liquid, and relative sugar content.

### FIELD

This invention relates to monitoring systems and more particularly to a system for monitoring the progression of the fermentation and brewing process of alcohol-based beverages.

### BACKGROUND

The process of producing an alcoholic beverage such as beer or wine is time consuming and difficult to perform in a consistent manner. Regarding the steps of brewing, Brewers and Vintners monitor this process using various tools such as hydrometers, thermometers, as well as their own sensory perception of taste, smell, and appearance. Existing processes often require samples be taken from the vessel or vat in which production is being performed, making measurements of the sample, looking at the sample, tasting the sample, smelling the sample, etc. Each sample reduces the volume of produced product and introduces more air into the vessel.

Problems with the existing sampling method includes the slight volume reduction noted, the opportunity of injecting unwanted materials into the process such as foreign yeasts, relatively long inter-sample periods, lack of consistency of each evaluation, lack of accountability/feedback/tracking, and the time expended by the brewer/vintner.

When samples are taken, often the extracted liquid is replaced by air which enables entry of foreign materials into the process, for example, foreign yeasts.

The time between samples (inter-sample period) is often long as it is not desired to remove too much product during the production process. Often, this leads to inconsistent product as a key brewing/vinting parameter might change early in the inter-sample period, affecting the overall resulting product.

There is often a lack of consistency due to inconsistent evaluations. The opinions and observations of one brewer/vintner often differs from another and often self-differs based upon moods, recent activities, recent food/drink consumption, etc. Further, in current times, a certain virus is well known to effect one's sense of taste and smell, often for months after recovery from the virus. Any brewer/vintner effected by such a virus or other ailments will not have the same perceptions as they previously had.

There is often a lack of accountability, feedback, and training provided to the brewer/vintner. In some processes, written notes are taken, but require the brewer/vintner to review pages of written notes if feedback is desired. Sharing of such written notes is often difficult. Further, as multiple batches from multiple vats are often combined, the available feedback is generally for the culminated process, not the individual processes performed on individual vessels.

What is needed is a system that will monitor various brewing/vinting parameters and provide data to the brewer/vintner that will help improve quality and consistency of the produced beverages.

### SUMMARY

In one embodiment, a system for reading and reporting data from a production of an alcoholic beverage is disclosed. The system includes a data capture device that has a sensor end. The sensor end is placed into a liquid and has at least two sensors for measuring sensor data related to the liquid. A processor located within the data capture device is operatively coupled to the at least two sensors and reads the sensor data from the at least two sensors. A transmitter located within the data capture device is operatively coupled to the processor. The processor periodically reads the sensor data related to the liquid from the at least two sensors and operates the transmitter to transmit the sensor data.

In another embodiment, a method for reading and reporting data from a production of an alcoholic beverage is disclosed. The method includes receiving a temperature from a first temperature sensor that is immersed in a liquid. The method further includes illuminating the liquid by a light source and receiving a plurality of brightness values from a light sensor that is interfaced to the liquid; each brightness value is associated with a wavelength of light. A relative sugar content level of the liquid is determined by comparing one brightness value (e.g., that of blue light) of the plurality of brightness values to at least one other brightness value of the plurality of brightness values and a clarity of the liquid is determined based upon the plurality of brightness values. The relative sugar content level, the clarity, and the temperature are displayed or reported to a user.

In another embodiment, a method for reading and reporting data from a production of an alcoholic beverage is disclosed. The method includes illuminating a liquid by a light source and receiving a plurality of brightness values from a light sensor that is interfaced to the liquid; each brightness value associated with a wavelength of light. As sugar in the liquid attenuates a first brightness value associated with a first wavelength of the light (e.g., blue), a relative sugar content level of the liquid is determined by comparing the first brightness value to at least one other brightness value of the plurality of brightness values.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a data connection diagram of the system for monitoring fermentation.

FIG. 2 illustrates a schematic view of a typical cell phone used in the system for the system for monitoring fermentation.

FIG. 3 illustrates a schematic view of a typical computer system such as a server or micro-controller.

FIGS. 4A, 4B, 4C, and 4D illustrate exemplary cell phone user interfaces of the system for monitoring fermentation.

FIG. 5 illustrates a plan view of a data capture device of the system for monitoring fermentation.

FIG. 6 illustrates block diagram of the data capture device of the system for monitoring fermentation.

FIG. 7 illustrates perspective diagram of one physical embodiment of the data capture device of the system for monitoring fermentation.

FIG. 8 illustrates a plan view of the sensor end of the data capture device of the system for monitoring fermentation using reflected light.

FIG. 9 illustrates a plan view of the sensor end of the data capture device of the system for monitoring fermentation using transmissive light.

FIG. 10 illustrates a block diagram of an operating embodiment of the system for monitoring fermentation.

FIG. 11 illustrates a data flow diagram shown in simplified form.

FIGS. 12-14 illustrate flowcharts of an embodiment of the system for monitoring fermentation.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

In general, the system for monitoring fermentation provides capabilities to measure fermentation parameters from a liquid in a vat such as a fermentation tank. In some embodiments, the liquid is must or any mixture used to produce an alcohol-based product such as beer or wine. As such vats are often opaque with limited access, in the past, the brewer/vintner would take samples of the liquid to determine progression of the fermentation progress. Taking of such samples reduces the volume of liquid in the vat, replacing the sampled liquid with air which is often undesired in the fermentation process, as air includes yeast spores of yeast varieties that are not controlled by the brewer/vintner. The system for monitoring fermentation provides data to the brewer/vintner as to temperature, clarity, and sugar content of the liquid in the vat to help the brewer/vintner make decisions during the fermentation process such as when to stop fermentation.

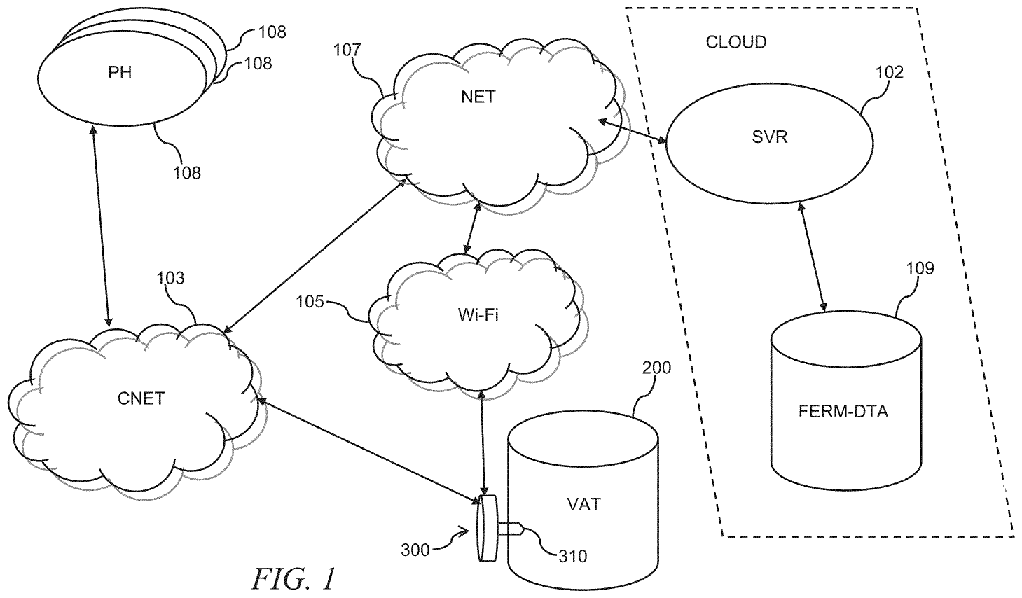

Referring to FIG. 1 illustrates a data connection diagram of the system for monitoring fermentation. In this example, one or more remote devices such as cell phones 108 communicate through the cellular network 103 and/or through a wide area network 107 (e.g., the Internet) to a server computer 102 (e.g., processing within the “cloud”).

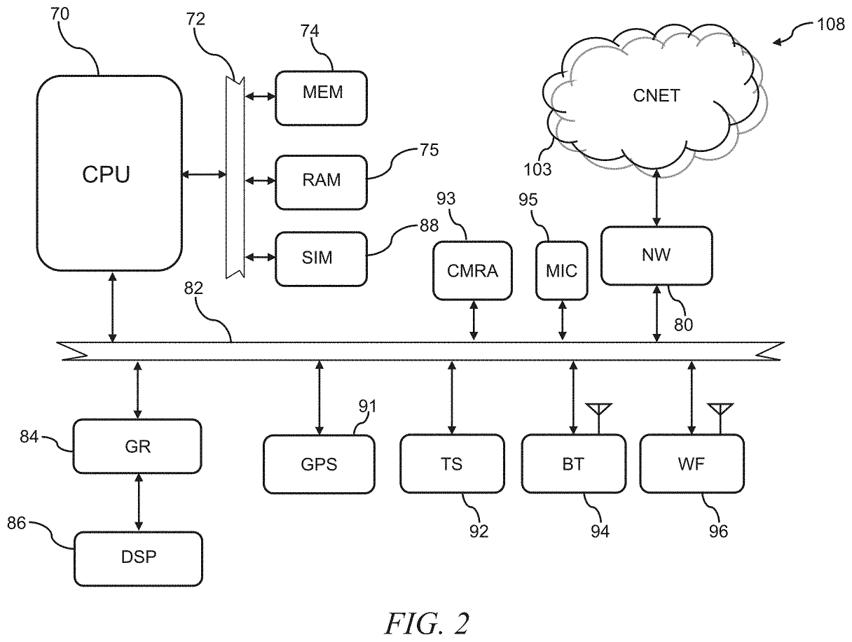

The server computer 102 has access to data storage 109 (e.g., cloud-based storage) for storing various data, including historical sensor data from one or more data capture devices 300, etc. Although one path between the remote devices or cell phones 108 and the server computer 102 is through the cellular network 103 and the wide area network 107 as shown, any known data path is anticipated. For example, the Wi-Fi transceiver 96 (see FIG. 2) of the remote devices or cell phone 108 is used to communicate directly with the wide area network 107, which includes the Internet, and, consequently, with the server computer 102.

The server computer 102 transacts with the remote devices or cell phones 108 through the network(s) 103/107 to present menus, to/on the remote devices or cell phones 108, provide data to the remote devices or cell phones 108, and to communicate information such as alerts to the remote devices or cell phones 108.

The server computer 102 transacts with applications running on the remote devices or cell phones 108 and/or with standardized applications (e.g., browsers) running on the remote devices or cell phones 108.

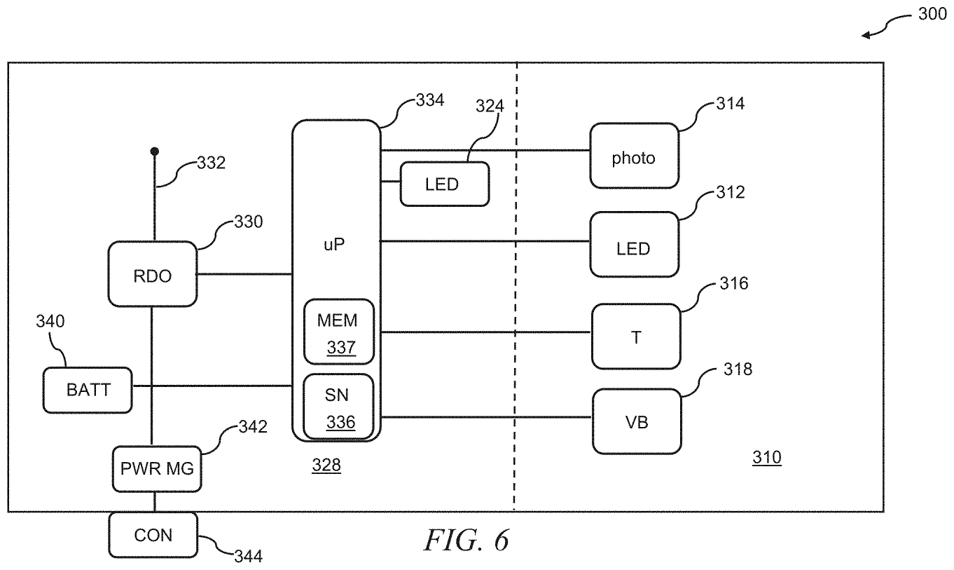

The system for monitoring fermentation includes at least one data capture device 300. During operation, each data capture device 300 is physically interface to a vat 200 such that the sensor end 310 of each data capture device 300 is in contact with or immersed in the liquid 210 that is within the vat. A seal prevents leakage of the vat where the data capture device 300 is installed. The sensor end 310 of the data capture device 300 is in fluid communication with the liquid 210 in the vat for taking readings regarding temperature, clarity, sugar content, etc. The sensor end 310 includes one or more sensors 314/316 (see FIG. 6) that are in fluid communications or are in contact with to the liquid 210. As such, the sensor end 310 is positioned such that the sensors 314/316 are able to measure various parameters of the liquid 210 such as temperature, clarity, etc.

Each data capture device 300 includes a transceiver 330 (see FIG. 6) that transmits messages to the server computer 102 through a wireless local area network 105 (e.g., a Wi-Fi network) and/or through the cellular network 103. Although any current or future wireless technology is anticipated, in some embodiments, the transceiver 330 communicates with a wireless local area network 105 (e.g., a local Wi-Fi network), which then connects to the server computer 102, for example, through any network fabric such as a wide area network 107 (e.g., the Internet).

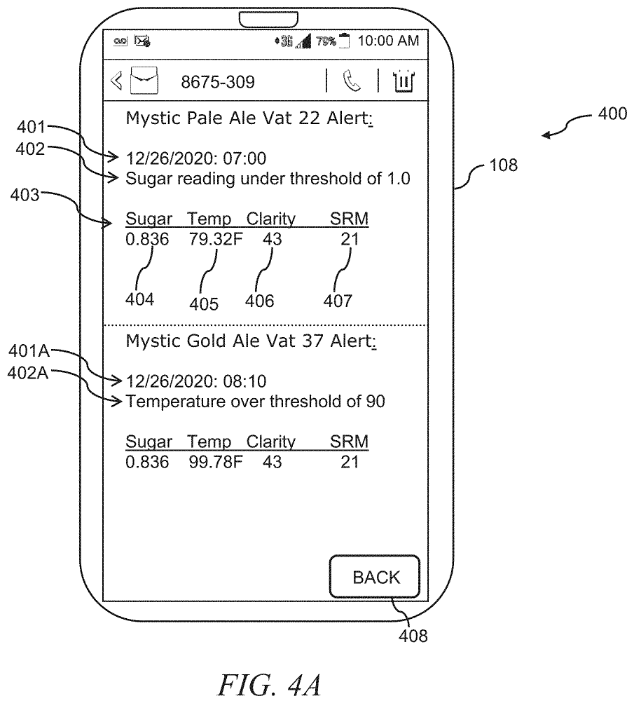

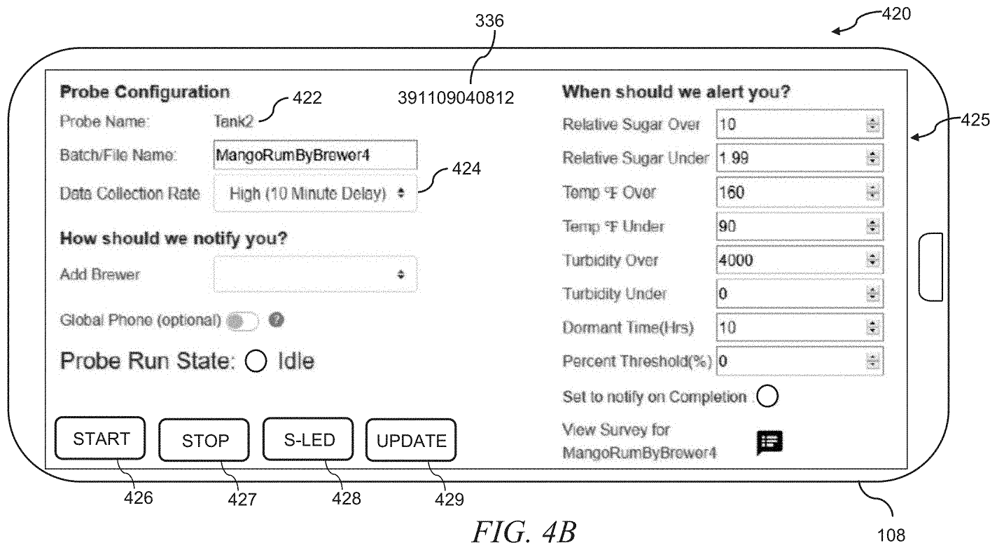

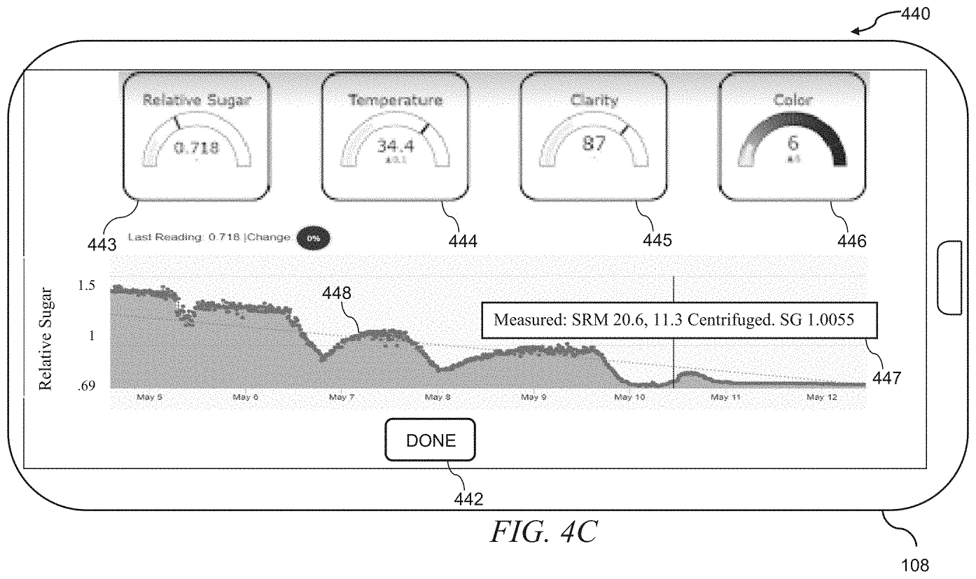

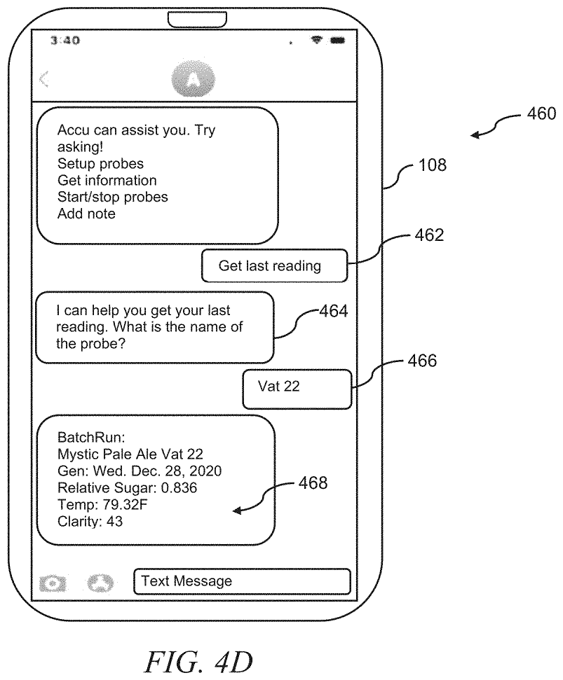

Referring to FIG. 2, a schematic view of a typical cell phone 108 is shown. The example cell phone 108 represents a typical cell phone used for accessing user interfaces (e.g., see FIGS. 4A, 4B, 4C, and 4D) of the system for monitoring fermentation, though it is fully anticipated that any computing device be used to access user interfaces of the system for monitoring fermentation. This exemplary cell phone 108 is shown in a typical form. Different architectures are known that accomplish similar results in a similar fashion and the present invention is not limited in any way to any particular cell phone 108 system architecture or implementation. In this exemplary cell phone 108, a processor 70 executes or runs programs in a random-access memory 75. The programs are generally stored within a persistent memory 74 and loaded into the random-access memory 75 when needed. Also accessible by the processor 70 is a SIM card 88 (subscriber information module) having a subscriber identification and often persistent storage. The processor 70 is any processor, typically a processor designed for phones. The persistent memory 74, random-access memory 75, and SIM card are connected to the processor by, for example, a memory bus 72. The random-access memory 75 is any memory suitable for connection and operation with the selected processor 70, such as SRAM, DRAM, SDRAM, RDRAM, DDR, DDR-2, etc. The persistent memory 74 is any type, configuration, capacity of memory suitable for persistently storing data, for example, flash memory, read only memory, battery-backed memory, etc. In some cell phones 108, the persistent memory 74 is removable, in the form of a memory card of appropriate format such as SD (secure digital) cards, micro-SD cards, etc.

Also connected to the processor 70 is a system bus 82 for connecting to peripheral subsystems such as a cellular network interface 80, a graphics adapter 84 and a touch screen interface 92. The graphics adapter 84 receives commands from the processor 70 and controls what is depicted on a display image on the display 86. The touch screen interface 92 provides navigation and selection features.

In general, some portion of the persistent memory 74 and/or the SIM card 88 is used to store programs, executable code, phone numbers, contacts, and data, etc. In some embodiments, other data is stored in the persistent memory 74 such as audio files, video files, text messages, etc.

The peripherals are examples and other devices are known in the industry such as a Global Positioning Subsystem 91, speakers, microphones, USB interfaces, Bluetooth transceiver 94, Wi-Fi transceiver 96, camera 93, microphone 95, image sensors, etc., the details of which are not shown for brevity and clarity reasons.

The cellular network interface 80 connects the cell phone 108 to the cellular network 103 through any cellular band and cellular protocol such as GSM, TDMA, LTE, etc., through a wireless medium. There is no limitation on the type of cellular connection used. The cellular network interface 80 provides voice call, data, and messaging services to the cell phone 108 through the cellular network.

For local communications, many cell phones 108 include a Bluetooth transceiver 94, a Wi-Fi transceiver 96, or both. Such features of cell phones 108 provide data communications between the cell phones 108 and data access points and/or other computers such as a server computer 102.

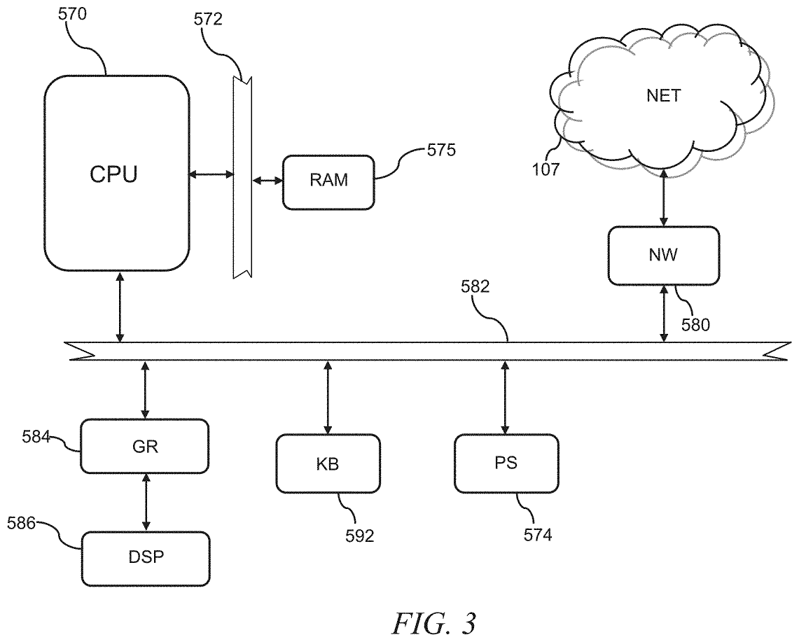

Referring to FIG. 3, a schematic view of a typical computer (e.g., server computer 102 which is anticipated to be one or more computing elements in the “cloud”) is shown. The example computer system represents a typical computer system used for back-end processing of sensor data, generating reports, displaying data, etc. This exemplary computer system is shown in its simplest form. Different architectures are known that accomplish similar results in a similar fashion and the present invention is not limited in any way to any particular computer system architecture or implementation. In this exemplary computer system, a processor 570 executes or runs programs in a random-access memory 575. The programs are generally stored within a persistent memory 574 and loaded into the random-access memory 575 when needed. The processor 570 is any processor, typically a processor designed for computer systems with any number of core processing elements, etc. The random-access memory 575 is connected to the processor by, for example, a memory bus 572. The random-access memory 575 is any memory suitable for connection and operation with the selected processor 570, such as SRAM, DRAM, SDRAM, RDRAM, DDR, DDR-2, etc. The persistent memory 574 is any type, configuration, capacity of memory suitable for persistently storing data, for example, flash memory, read only memory, battery-backed memory, magnetic memory, etc. The persistent memory 574 is typically interfaced to the processor 570 through a system bus 582, or any other interface as known in the industry.

Also shown connected to the processor 570 through the system bus 582 is a network interface 580 (e.g., for connecting to a data network or wide area network 107), a graphics adapter 584 and a keyboard interface 592 (e.g., Universal Serial Bus—USB). The graphics adapter 584 receives commands from the processor 570 and controls what is depicted on a display image on the display 586. The keyboard interface 592 provides navigation, data entry, and selection features.

In general, some portion of the persistent memory 574 is used to store programs, executable code, data, contacts, and other data, etc.

The peripherals are examples and other devices are known in the industry such as speakers, microphones, USB interfaces, Bluetooth transceivers, Wi-Fi transceivers, image sensors, temperature measuring devices, etc., the details of which are not shown for brevity and clarity reasons.

Referring to FIGS. 4A, 4B, 4C, and 4D, an exemplary cell phone user interface of the system for monitoring fermentation is shown. Note that any user interface is anticipated, operating on any device, for example, operating on a cell phone 108.

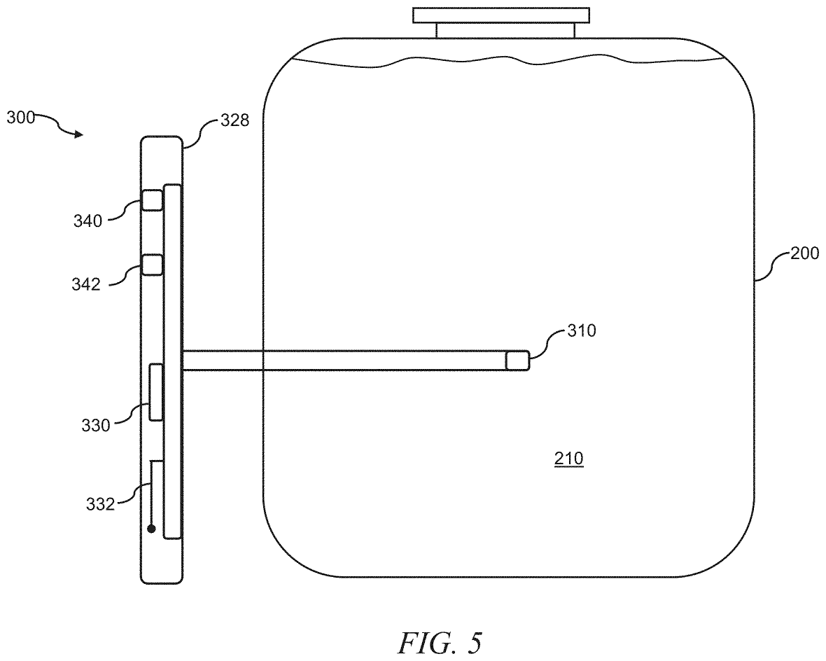

Although many user interfaces are anticipated, one example user interface is a text message user interface 400 that is used to report processed data derived from data received from the data capture device 300 (see FIGS. 5 and 6). The text message user interface 400 runs on a cell phone 108 or any other device. When a messaging application runs, for example, on the user's cell phone 108, the messaging application communicates with the cellular network 103 and receives short message service (SMS) messages from the server computer 102. Such messages include measurements and alerts. In this example, an alert has been received indicating that the sugar reading of the liquid 210 that is in a vat 200 (“Mystic Pale Ale Vat 22”) is below a preset threshold 402; the alert includes the date/time 401 of the measurement (7:00 on 12/26/2020). Further in this example, a second alert 402A has been received indicating that the liquid 210 in a vat 200 (“Mystic Gold Ale Vat 37”) has exceeded a preset temperature limit of 90 degrees, again including the date/time 401A of the alert (8:10 on 12/26/2020). In some embodiments, once the alert is tended to, a clear operation 406 is invoked. Note that the display names of the vats 200 (e.g., “Mystic Pale Ale Vat 22”) are created during configuration of each data capture device 300, mapping a human-understandable name to a serial number 336 (see FIG. 6) of the respective data capture device 300.

It is anticipated that, in any of the user interfaces (e.g., those for FIGS. 4A, 4B, 4C, or 4D), there are provisions for making and reading back notes. This feature, called BrewBook, provides the brewer with a mechanism to take real-time shareable notes that can be viewed by all interested parties.

Note that SRM stands for “Standard Reference Method,” a parameter used to measure the relative darkness of a beer and “Clarity” is a measure of the degree to which the water loses its transparency due to the presence of suspended particulates.

FIG. 4A shows another user interface 420 that is used to administer a data capture device 300. In this example, the serial number 336 of the data capture device 300 is displayed. The user has entered a friendly name 422 for the associated vat 200 as well as a name of the liquid 210 being produced in this vat 200. The user is able to set alarms, for example, an alarm if the relative sugar reading is over 10 or the temperature falls below 90 F. There are also directives for starting 426 this data capture device 300, for stopping 427 this data capture device 300, for suppressing 428 the indicator 324 of this data capture device 300, and for updating 429 this data capture device 300.

In FIG. 4C, an exemplary data display user interface 440 is shown. Any type of data representation and display are anticipated and the disclosed user interfaces do not, in any way, limit the present application.

In this example, current reading for relative sugar 443, current temperature 444, current clarity 445, and current color 446 are displayed across the top along with one or more charts 448 showing progress or change over a period of time. In this example, only one chart 448 reporting relative sugar content is shown for brevity and clarity reasons, though it is equally anticipated to show a chart 448 for any of the measured and/or calculated readings (e.g., relative sugar content, sugar content, clarity, temperature, color, specific gravity, alcohol-by-volume). In this embodiment, the user has had a sample of the liquid 210 analyzed and the actual laboratory measurements 447 are shown. Note that the chart 448 for relative sugar content is following the normal progression of lower amounts of sugar over time as the yeast is converting the sugar into alcohol.

FIG. 4D shows an artificial-intelligence (AI) driven user interface 460. Instead of a fixed user interface as shown prior, the artificial-intelligence (AI) driven user interface 460 accepts natural language requests such as “get last reading” 462. The artificial-intelligence (AI) driven user interface 460 then interprets the request and acts. In this example, the user has not provided enough information to act on the request and the artificial-intelligence (AI) driven user interface 460 has asked for the missing information, for example, “what is the name of the probe?” 464. If the user already included the name of the probe in the original request, this step is not needed. The user has entered “vat 22” 466 as the name of the probe and the artificial-intelligence (AI) driven user interface 460 responds with current readings 468 for that vat.

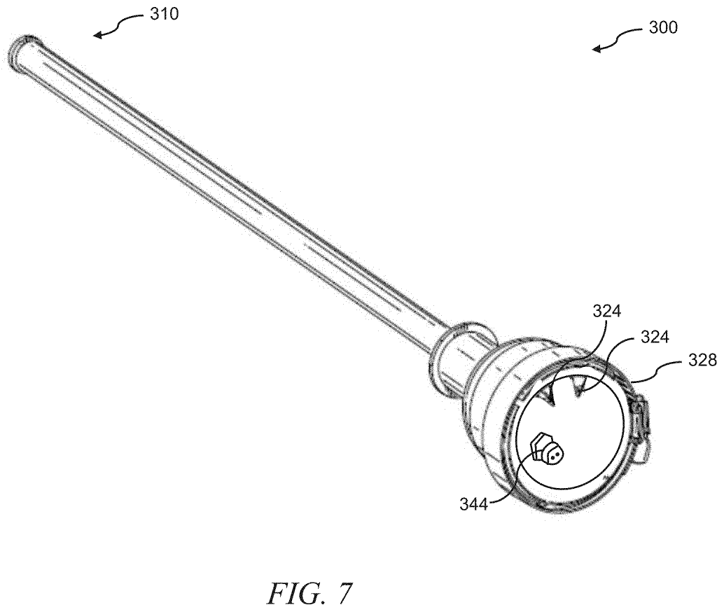

Referring to FIGS. 5 through 9, data capture devices 300 are shown. Each data capture device 300 includes a processor 334 and a transceiver 330 (or transmitter) that transmits messages to the server computer 102 through a wireless local area network or, in some embodiments, directly through the cellular network 103. Note that it is known that equivalent implementations be made using logic instead of a processor 334. In some embodiments, the processor is an ARM processor such as the Cortex M3 ARM processor. Note that some processors 334 are embedded with circuitry that provide enhanced input/output such as analog-to-digital converters while other processors 334 require external circuitry to perform such functions.

In some embodiments, the transceiver 330 is a Wi-Fi (802.11) transceiver 330 that is paired with a local Wi-Fi network at the alcoholic beverage production facility, though any transceiver 330 is anticipated, including a transceiver 330 that communicates directly with the cellular network 103. Each data capture device 300 communicate to the server computer 102 (e.g., cloud computing system) over any network fabric, wired or wireless or combination of both. The data capture devices 300 communicate using any known wireless frequency and protocol, including directly through the cellular network 103 or wide area network 107 using any wireless protocols such as LTE, 3G, 4G, 5G, using 802.11 (Wi-Fi), using Bluetooth, or any combination thereof, etc. An antenna 332 that is appropriate for the transceiver 330 is electrically connected to the transceiver 330.

In some embodiments, the data capture devices 300 include a backup battery 340 to continue operations during a power failure. In such embodiments, when a power failure is detected, capturing of data continues but the transceiver 330 is disabled (low power state) and instead of transmitting the data, the data is stored locally in memory of the processor 334 and the data is later transmitted when power is again detected.

The processor 334 of each data capture devices 300 has a unique serial number 336 (either internal to the processor 334 or an external device electrically interfaced to the processor 334). The unique serial number 336 is communicated in each transaction to the server computer 102 to identify that particular data capture device 300. In some embodiments, the server computer 102 translates the unique serial number 336 into a user-friendly name (e.g., “Mystic Pale Ale Vat 22”).

At the sensor end 310 of each data capture device 300 are one or more sensors 314/316 (two sensors 314/316 are shown). In the data capture device 300 of FIG. 6, there is a light sensor 314 and a temperature sensor 316. Used in conjunction with one or more light emitting diodes 312, the light sensor 314 provides measurements that are proportional to the clarity of the liquid 210 in which the sensor end 310 is immersed.

In some embodiments, the light emitting diode(s) 312 emit a white light at a specific color temperature (e.g., measured on a Kelvin scale). In such embodiments, the light sensor 314 differentiates intensity (e.g., measured in Lux, a measurement of luminous flux per unit area) by color, for example, reporting an intensity for each of the primary colors of red, green, and blue. In some embodiments, the light sensor 314 includes compensation for the light emitting diode 312 being used. For example, if the light emitting diode 312 emits a warm white light, then such compensation reduces the red measurement while if the light emitting diode 312 emits a cool white light, then the compensation reduces the blue measurement. In some embodiments, a compensation table is provided to match and normalize the measurement from the light sensor 314 to the color temperature of the light emitting diode 312.

In an alternate embodiment, the light emitting diode(s) 312 emit colors of light, for example, by having red, blue, and green emitters. In such embodiments, the light sensor 314 measures only intensity (e.g., measured in Lux, a measurement of luminous flux per unit area), not by color. In this embodiment, as an example, to measure an intensity for each of the primary colors of red, green, and blue, each emitter is sequentially energized while reading the intensity of that color from the light sensor 314.

In some embodiments, the light emitting diode(s) 312 emit other wavelengths of light that are detected by the light sensor 314. For example, to measure alcohol-by-volume (ABV), one of the light emitting diodes 312 emits near infrared (NIR) and the light sensor 314 reads near infrared (NIR). In this example, alcohol in the liquid 210 absorbs near infrared light and by measuring the amount of near infrared light passed through the liquid, an approximation of the alcohol content by volume is made (ABV). It is fully anticipated that any wavelength of light be emitted by the light emitting diode(s) 312 and, similarly, detected by the light sensor 314. It is also anticipated that the light sensor 314 comprise one or more independent light detectors, each measuring light of a different wavelength. For example, in one embodiment, the light sensor 314 has four light detectors, one light detector for red, one light detector for green, one light detector for blue, and one light detector for near infrared.

In some embodiments, the light emitting diode(s) 312 are controlled by output ports of the processor 334 to illuminate a selected light emitting diode 312 (e.g., specific wavelength or wavelengths of light) at a selected intensity when a measurement is taken. For example, blue light is absorbed by sugar so by emitting blue light and then white light (or other color) and measuring corresponding brightness of each as received by the light sensor 314; then comparing the brightness levels provides a relative measurement of sugar content in the liquid 210. To further this, when blue light is emitted from the light emitting diodes 312, the amount of light reaching the light sensor 314 is proportional to the amount of sugar in the liquid 210. When white light is emitted from the light emitting diodes 312, the amount of light reaching the light sensor 314 is proportional to the overall clarity of the liquid 210. Again, in some embodiments, the light emitting diode(s) emit white light and the light sensor 314 provides readings of light received in two or more spectrums of light (e.g., red, green, and blue). In some embodiments, the light emitting diode 312 (white) is turned on prior to making the measurement and turned back off after making the measurement to conserve power and reduce heat transmission into the liquid 210.

As will be discussed, the reading from the light sensor 314 related to white light provides a relative measurement of clarity and the reading from the light sensor 314 related to blue light provides a relative measurement of sugar content. In some embodiments, both are used to derive the sugar content of the liquid 210, which is an important measurement in the production of alcoholic beverages. By measuring the white light absorption, a clarity baseline is determined. Then, by measuring the blue light absorption, a sugar clarity is determined. As an example, if almost zero white light is measured at the light sensor 314, then the liquid 210 is very dark and measuring blue light absorption will not be useful; but if 50% white light is measured and 40% blue light is measured, then from such, a relative sugar content is determined.

In some embodiments, a vibration device 318 is integrated into the sensor end 310, for example, a micro-motor with an off-set weight affixed to a shaft of the micro-motor or a piezo-electric vibration device. As the liquid 210 often has particulate of solid matter or high-viscosity material that sometimes gets stuck between the light sensor 314 and the light emitting diode 312 or particulate mater sticking to the optional window 315, the vibration device 318 will purge such matter from the sensor end 310 before reading of clarity using the light sensor 314. In such embodiments, the vibration device 318 is controlled by the processor 334 to initiate vibration before taking measurements.

It is anticipated that in some embodiments, the processor 334 includes input ports, output ports, and one or more analog-to-digital converters. The analog-to-digital converters convert analog inputs (e.g., from the sensors 314/316) into digital values. Each of the sensors 314/316 are electrically interfaced to the processor 334 of each data capture device 300 such that an analog input (e.g., analog-to-digital converter and/or a multiplexer connected to a single analog-to-digital converter) of the processor 334 periodically reads the current value of each sensor 314/316 to determine clarity, temperature, etc. Note that, in some embodiments, the light sensor 314 converts the analog light measurement(s) into digital and provides a digital value of the light measurement(s) to an input of the processor 334.

It should be noted that any analog or digital interface between the processor 334 and the sensors 314/316 is anticipated and included here within.

In some embodiments, the processor 334 pre-processes the readings from the sensors 314/316 to reduce the size of the data transmission from the transceiver 330 to the server computer 102.

In some embodiments, one or more indicators 324 on the body 328 of the data capture device 300 provide status of operation a power.

Power is provided by an external power source (e.g., a standard 5V power supply), entering the body 328 of the data capturing device 300 through a connector 344 and regulated/conditioned by a power management circuit 342 that also controls charging of the backup battery 340.

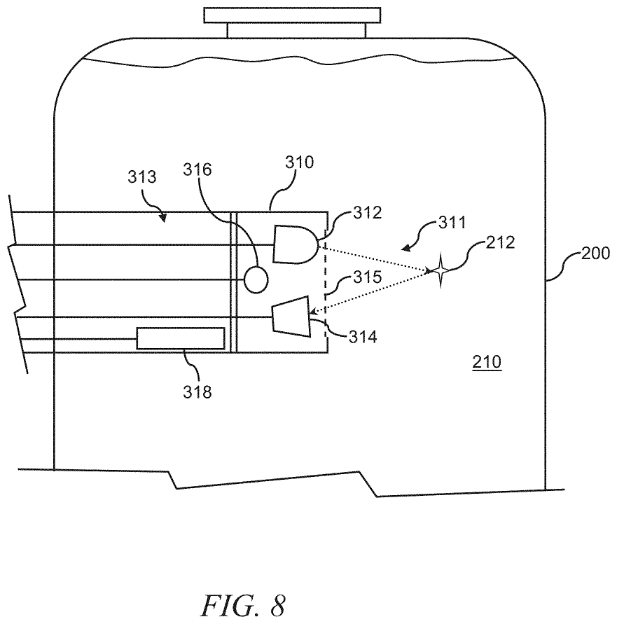

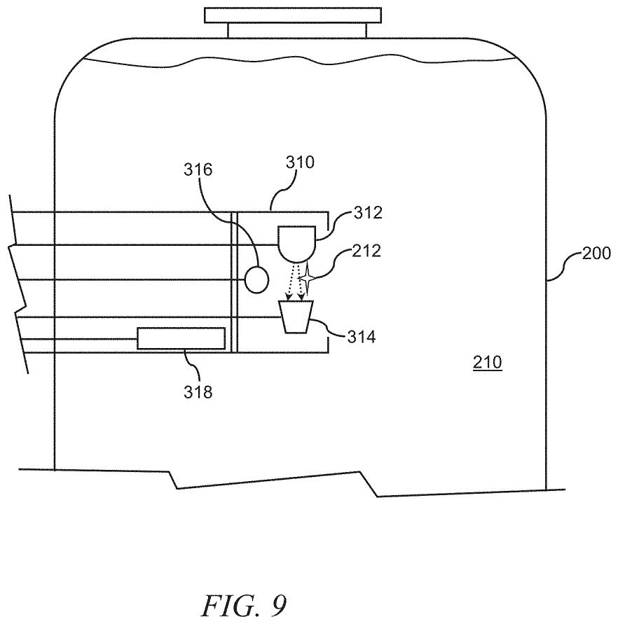

In FIGS. 8 and 9, expanded views of the sensor end 310 are shown. In both FIG. 8 and FIG. 9, the optional vibration device 318 is shown (preferably located in the dry area 313 of the sensor end 310). The tip of the sensor end 310 is open, allowing the liquid 210 to enter the wet area 311 of the sensor end 310. The temperature sensor 316 is anticipated to be mounted in either the wet area 311 or in the dry area 313 and thermally interfaced to the liquid 210.

In some embodiments, a window 315 allows transmission of light while preventing liquids from entering the tip of the sensor end 310. Although it is anticipated that the window 315 be made of any clear or translucent material, one such material is glass.

In FIG. 8, reflectivity is used to measure the liquid 210. In this, the light emitting diode(s) 312 emit light outwardly into the liquid 210 and as the light strikes particles 212 in the liquid 210, the light reflects back and is received by the light sensor 314. In this example, if the liquid 210 is very clear, very little light is reflected back to the light sensor 314.

In FIG. 9, transparency is used to measure the liquid 210. In this, the light emitting diode(s) 312 emit light through the liquid 210 and towards the light sensor 314. As the light strikes particles 212 in the liquid 210, the particles 212 block light received by the light sensor 314. In this example, if the liquid 210 is very clear, more light is received by the light sensor 314.

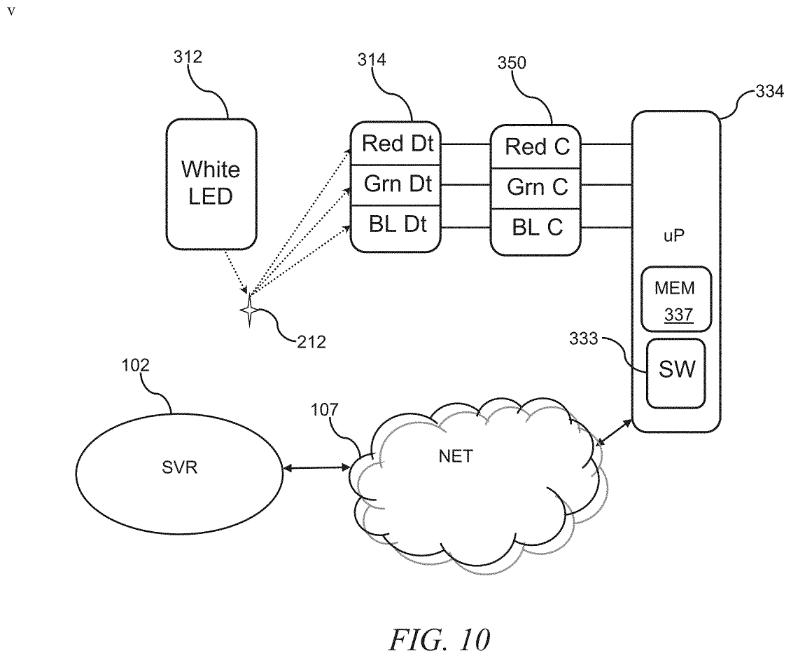

Referring now to FIG. 10, a block diagram of an operating embodiment of the system for monitoring fermentation for the production of an alcoholic beverage. This example uses the embodiment of FIG. 8 for clarity and brevity as it is equally anticipated to utilize transparency as in the scenario of FIG. 9 instead of reflectivity of the scenario of FIG. 8.

In this example, the light emitting diode 312 emits a white light of a known kelvin temperature into the liquid 210. A certain amount of the white light reflects back from the liquid 210 (e.g., reflecting back from particulates 212) and onto the light sensor 314 which, in this example, is an array of three light detectors, one for red light, one for green light, and one for blue light. Note that in some embodiments, more or fewer light detectors are provided, including two red detectors, two blue detectors, two green detectors, and two clear detectors. It is also anticipated that there are more than one light emitting diode 312, emitting white light or any spectrum of light. Note that when the white light passes through the liquid 210, certain wavelengths of the white light are attenuated by individual compositions of the liquid 210. For example, light in the blue spectrum is attenuated by sugar suspended in the liquid 210. Therefore, the higher the sugar content of the liquid 210, the lower amount of blue light that will reach the light sensor 314. Likewise, lower sugar content in the liquid 210 results in higher amounts of blue light reaching the sensor 314. The relative amounts of blue light received at the light sensor 314 are used to provide a relative sugar content reading for the liquid 210.

Note that although the light emitting diode 312 typically emits a very broad spectrum of light consistently throughout the life of the light emitting diode 312, white light emitting diodes 312 don't all emit the same spectrum of white light, some being warmer or cooler (having different Kelvin temperatures). To compensate for the specific light emitting diode 312, in some embodiments a compensation table 350 is provided, either as part of the light emitting diode 312, between the light emitting diode 312 and the processor 334, or as a function of the processor 334. The compensation table 350 adjusts the light values of each color (e.g., red/green/blue) to compensate for the light output of the light emitting diode 312. For example, if the light emitting diode 312 selected produces a warm white light (e.g., more red light than green and blue) then the compensation table 350 discounts the detected red light. In this, if the light output of the light emitting diode is 1100 lux of red, 1000 lux of green and 1000 lux of blue, then the compensation table 350 will reduce the reading for red by 10% or increase the readings for green and blue each by 10% to compensate for the light emitting diode 312.

Software 333 that runs on the processor 334 receives the color readings (and other sensor readings). If wireless communications is available, the readings are periodically forwarded through the network(s) 107 to the server computer 102. Note that if wireless communication is not available, one or more color readings (and other sensor readings) are time-stamped and stored in the memory 337 associated or included with the processor 334, and when wireless communication is re-established, the readings, serial number 336, and time stamps are forwarded to the server computer 102 for processing.

The server computer 102 then stores the color readings, serial number 336 (or any identifier), time stamps and other sensor readings and further processes the color readings and other sensor readings to present data to the remote devices of the user (e.g., cell phone 108).

The processing includes comparing the lux received for each color to extract clarity and relative sugar content. For example, if the liquid 210 is as clear as distilled water, then there will be very little light reflected back onto the light sensor 314, indicating a high level of clarity. In another example, if the liquid is not clear, some amount of red/green/blue light will reflect back onto the light sensor 314. If there is a high amount of sugar content in the liquid, less blue light will make it to the light sensor 314 as sugar absorbs blue light more than red and green. The relative lux received for each color (red/blue/green) is also used to determine the color of the liquid. For example, in brewing beer, there are different colors desired such as amber, brown, dark brown, etc., and each specific color of beer will absorb each color (red/green/blue) differently. Using the relative lux received for each color (red/green/blue) is used to determine the color of the liquid 210. As an example, if the liquid color is predominantly red, then the red light will be reflected more than the blue and green light as received by the light sensor 314.

A note should be made that although this description includes three primary colors of red, green, and blue, there is no restriction on the light wavelengths emitted by the light emitting diode 312 and single or multiple wavelengths detected by the light sensor 314, nor the mechanism used to distinguish colors such as filters or prisms, etc. Further, the use of non-visible (to humans) wavelengths of light is fully anticipated including all wavelengths of infrared as well as ultraviolet.

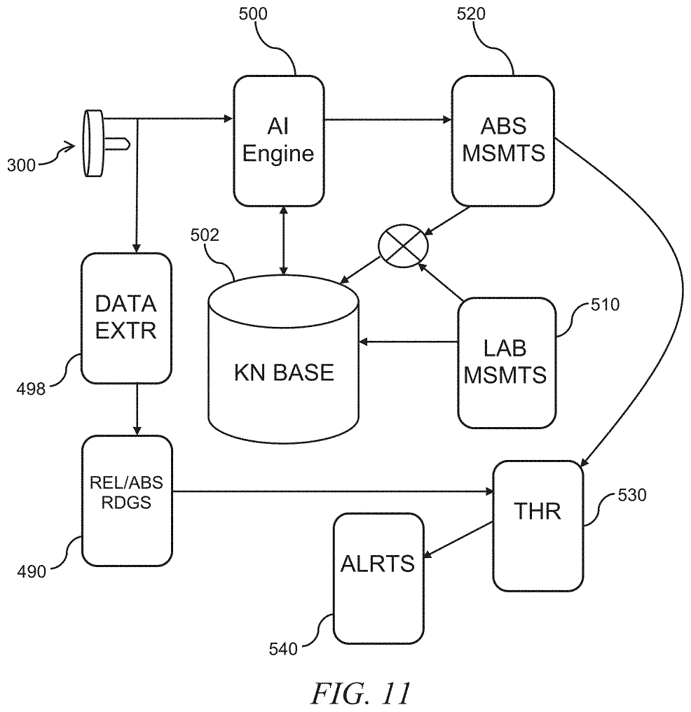

In FIG. 11, a data flow diagram is shown in simplified fashion. Data from the data capture device 300 is processed by software running on the server computer 102 by a data extraction module 498 to perform steps similar to above (e.g., the data extraction module 498 processes the color data received from the light sensor 314 and extracts from that, for example, relative sugar content and color of the liquid 210) and to generate relative and/or absolute readings 490. For example, as temperature is measured with a temperature sensor 316 that directly measures absolute temperature, this is an absolute reading while as sugar content is derived from color data, this is a relative reading.

Relative sugar content and color are important to alcoholic beverage makers to monitor the process, knowing that the relative sugar content decreases as the yeast converts the sugars to alcohol, but absolute numbers are needed to know exactly when the process is complete. In the past, brewers sent samples of the liquid 210 to a laboratory where the liquid 210 is analyzed to determine the exact sugar content, alcohol content, color, specific gravity, etc.

To approximate absolute readings, the color data from the light sensor 314 is processed by an artificial intelligence engine 500. The artificial intelligence engine 500 has a knowledge base 502 that has data from prior color data readings as they relate to laboratory measurements 510. For example, if at 3PM on Jan. 1, 2021, a sample of the liquid 210 was taken and sent for analysis, when the laboratory measurement 510 are available, the laboratory measurements 510 are correlated to the color/temperature data received from the data capture device close to 3PM on Jan. 1, 2021. In that way, the artificial intelligence engine 500 adjusts decisions about what the color data represents in absolute terms based upon the laboratory measurements 510. Further, the artificial intelligence engine 500 is able to better determine the color of the liquid 210 using laboratory measurements 510 for color (e.g., SRM or Standard Reference Method) or color as observed by a trained technician.

The output of the artificial intelligence engine 500 is then approximated absolute measurements 520. As the knowledge base is fed with more measurements that correspond to laboratory results that are provided by multiple users, the accuracy of the approximated absolute measurements 520 increases and, eventually, the accuracy will be very close to the laboratory measurements 510.

In some embodiments, thresholds 530 are set by each user for one or more data capture device 300. For example, a minimum temperature threshold is set by the user for one data capture device 300 or for a set of data capture devices 300. When data is received from the data capture device(s) 300 and the data is extracted 498 into relative and/or absolute readings 490, besides storing/reporting the relative and/or absolute readings 490, the relative and/or absolute readings 490 are compared to any thresholds 530 that were set by the user and, if any of the thresholds 530 are exceeded, one or more alerts 540 are generated (e.g., a text message is sent to the user's cell phone 108. Likewise, the absolute measurements 520 as generated by the artificial intelligence engine 500 are also compared to thresholds 530 and if any of the thresholds 530 are exceeded, one or more alerts 540 are generated (e.g., a text message is sent to the user's cell phone 108. For example, if a threshold 530 is set for temperature less than 68 F and data reported from one of the covered data capture devices 300 reports a temperature of 67 F, an alert 540 is triggered and the user is contacted, warning of which vat 200 and the measured value. Note that the term, “user,” is meant to be very broad and include any person or a device of any person having to do with the administration and operation of one or more data capture devices 300, for example, a technician, an owner, a brewer, a vintner, etc.

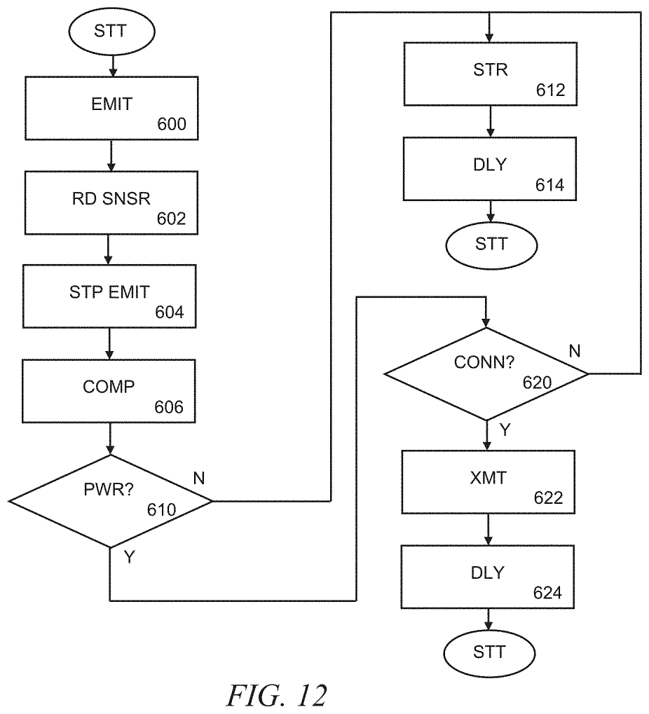

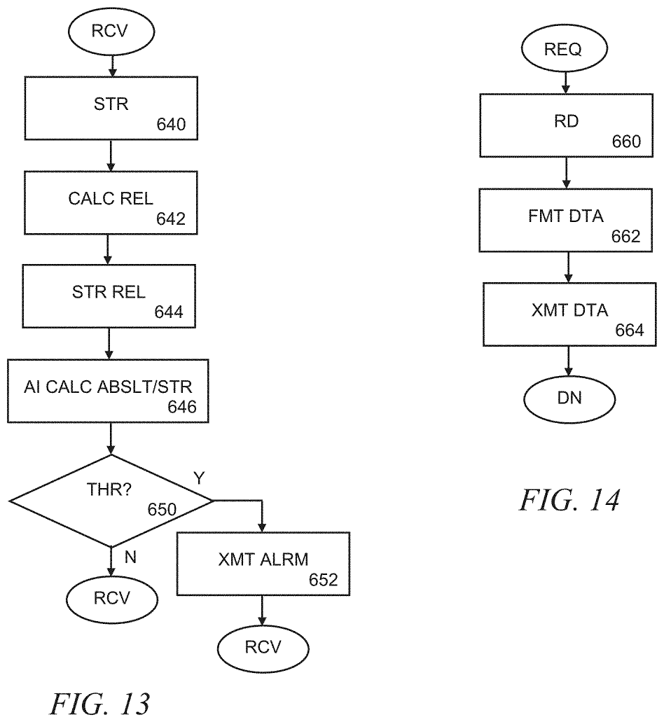

Referring to FIGS. 12-14, example flowcharts of the system for monitoring fermentation are shown. In FIG. 12, a sample of the software 333 that runs on the processor of the data capture device 300 is shown. At the start of each measurement cycle, light is emitted 600 from the light emitting diode(s) 312. In some embodiments, the light emitting diode(s) 312 are controlled by the processor to emit light of any selected color. The light sensor 314 and other sensors (e.g., the temperature sensor 316) are then read 602, the light sensor 314 reporting the amount of light in each monitored spectrum of light. In embodiments in which the processor controls the light emitting diode 312, the light emitting diode 312 is extinguished 604. The reported amount of light in each monitored spectrum of light is compensated 606 to normalize based upon the light emitting diode(s) 312 that are used. If power is off 610, the data from the sensors is time stamped and stored 612, then after a delay 614, the above is repeated. Note that once it is determined that power is back on, prior stored data is transmitted. Next, if there is no data connection 620, the data from the sensors is time stamped and stored 612, then after a delay 614, the above is repeated. If there is a connection 620, the data, serial number 336, (and optionally a time stamp) are transmitted 622, then after a delay 624, the above is repeated.

When the data, serial number 336 and optionally the time stamp are received at the server, the data, serial number 336 and optionally the time stamp are stored 640, the relative values are calculated 642 (e.g., relative sugar content) and stored 644. The absolute values are calculated 646 using the artificial intelligence engine 500 and stored as well. Now, thresholds are checked 650 for relative and/or absolute measurements/values exceeding any threshold (e.g., higher than 160 F) or being below any limit threshold (e.g., less than 68 F). If any thresholds checked 650 are exceeded, an alarm is transmitted 652 (e.g., a text message is sent to the user). The above then repeats.

In FIG. 14, a request is received at the server computer 102 for data regarding one or more of the data capture devices 300 (e.g., a text message is received from a user). The data is read 660 (e.g., from memory of the server computer 102 or a database of the server computer 102), formatted 662, and transmitted 664 back to the user.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for reading and reporting data from a production of an alcoholic beverage, the system comprising:
a data capture device, the data capture device having a sensor end for placement within a liquid, the sensor end having at least two sensors for measuring sensor data related to the liquid;
a processor located within the data capture device is operatively coupled to the at least two sensors for reading the sensor data from the at least two sensors;
a transmitter located within the data capture device and operatively coupled to the processor, the processor periodically reads the sensor data related to the liquid from the at least two sensors and the processor operates the transmitter to transmit the sensor data.

2. The system of claim 1, wherein the at least two sensors comprise a temperature sensor and a light sensor, the light sensor receiving light emitted from a light source that is within the sensor end, the light passes from the light source through the liquid and onto the light sensor.

3. The system of claim 2, wherein the light that passes from the light source is directly aimed at the light sensor, therefore the light sensor measuring clarity of the liquid.

4. The system of claim 2, wherein the light that passes from the light source is aimed away from the light sensor, therefore the light sensor measuring light reflected back from the liquid.

5. The system of claim 1, further comprising a vibration device interfaced to the sensor end of the data capture device, the vibration device for freeing accumulated debris from the liquid.

6. The system of claim 1, further comprising a server, the server receiving the sensor data related to the liquid and the server storing portions of the sensor data related to the liquid that are absolute and the server calculating relative measurements from the sensor data related to the liquid.

7. The system of claim 6, further comprising the server calculating a relative measurement of sugar content from the sensor data related to the liquid, the sensor data related to the liquid comprising brightness data for at least two colors of light and the server calculating the relative measurement of the sugar content based upon ratios calculated between the brightness data for the at least two colors of the light.

8. The system of claim 6, further comprising the server calculating an absolute measurement of sugar content from the sensor data related to the liquid, the sensor data related to the liquid comprising brightness data for at least two colors of light and the server calculating the absolute measurement of the sugar content by processing the brightness data of the at least two colors of the light with an artificial intelligence engine that has a knowledge base, the knowledge base includes a plurality of laboratory measurements from prior liquids that correspond to prior data related to the prior liquids.

9. A method for reading and reporting data from a production of an alcoholic beverage, the method comprising:
receiving a temperature from a first temperature sensor that is immersed in a liquid;
illuminating the liquid by a light source;
receiving a plurality of brightness values from a light sensor that is interfaced to the liquid, each brightness value associated with a wavelength of light;
determining a relative sugar content level of the liquid by comparing one brightness value of the plurality of brightness values to at least one other brightness value of the plurality of brightness values;
determining a clarity of the liquid based upon the plurality of brightness values; and
displaying the relative sugar content level, the clarity, and the temperature.

10. The method of claim 9, wherein the plurality of brightness values from the light sensor comprises three brightness values, a first brightness value of the three brightness values is for a red wavelength of the light, a second brightness value of the three brightness values is for a green wavelength of the light, and a third brightness value of the three brightness values is for a blue wavelength of the light.

11. The method of claim 10, wherein in the step of determining the relative sugar content level of the liquid comprises comparing the third brightness value to the first brightness value and the second brightness value as the blue wavelength of the light is absorbed by sugar.

12. The method of claim 10, further comprising determining an absolute sugar content level of the liquid using an artificial intelligence engine that is fed with a plurality of prior brightness values for a prior liquid that are coupled to corresponding laboratory measurements of the prior liquid.

13. The method of claim 9, further comprising vibrating the light sensor and the light source to dislodge solids received from the liquid.

14. A method for reading and reporting data from a production of an alcoholic beverage, the method comprising:
illuminating a liquid by a light source;
receiving a plurality of brightness values from a light sensor that is interfaced to the liquid, each brightness value associated with a wavelength of light;
sugar in the liquid attenuating a first brightness value associated with a first wavelength of the light;
determining a relative sugar content level of the liquid by comparing the first brightness value to at least one other brightness value of the plurality of brightness values.

15. The method of claim 14, wherein the first wavelength of the light is a blue wavelength.

16. The method of claim 15, wherein the at least one other brightness value of the plurality of brightness values comprises a second brightness value associated with a red wavelength of the light and a third brightness value associated with a green wavelength of the light.

17. The method of claim 16, further comprising determining a clarity of the liquid based upon the first brightness value, the second brightness value and the third brightness value.

18. The method of claim 16, further comprising determining a color of the liquid based upon the first brightness value compared to the second brightness value compared to the third brightness value.

19. The method of claim 16, further comprising determining an absolute sugar content level of the liquid using an artificial intelligence engine that is fed with a plurality pairs of prior brightness values for a prior liquid and corresponding laboratory measurements of the prior liquid.

20. The method of claim 14, further comprising vibrating the light sensor and the light source to dislodge solids received from the liquid.
